(19) United States
(12) Patent Application Publication
(10) Pub. No.: US 2003/0023131 A1
(43) Pub. Date: Jan. 30, 2003

(54) Apparatus and method for reducing heart pump backflow

(76) Inventor: James F. Antaki, Pittsburgh, PA (US)

Correspondence Address: MADSON & METCALF, GATEWAY TOWER WEST, SUITE 900, 15 WEST SOUTH TEMPLE, SALT LAKE CITY, UT, 84101

(21) Appl. No.: 10/163,257

(22) Filed: Jun. 5, 2002

Related U.S. Application Data
(60) Provisional application No. 60/296,393, filed on Jun. 6, 2001.

(51) Int. Cl. A61N 1/362

(52) U.S. Cl. 600/16

(57) ABSTRACT

An apparatus and method for reducing backflow in a heart pump device are disclosed. In one configuration, a ventricular assist device (VAD) has a pump connected to a ventricle of the heart by an inflow cannula and to a blood vessel by an outflow cannula. The VAD is designed in such a manner that the overall reactance of the VAD is comparatively high, thereby reducing the incidence of time-varied flows, such as backflow, through the VAD. Resistance of the VAD to steady state flow may simultaneously be maintained below a threshold level to provide low-power operation. The desired reactance and resistance properties of the VAD may be obtained by adjusting the reactance and resistance of one or both of the cannulae. The reactance of a cannula may be set at the desired level by tuning one or more geometric characteristics of the cannula, including the bore diameter, length, cross sectional bore shape, and compliance of the cannula.

### RELATED APPLICATIONS

This application claims the benefit of U.S. Provisional Application Serial No. 60/296,393, filed Jun. 6, 2001 and entitled HEART PUMP FLOW PATH DEVICE. The disclosure of the above application is incorporated herein by reference.

### BACKGROUND OF THE INVENTION

1. Field of the Invention

The present invention relates to systems and methods for augmenting coronary function. More specifically, the present invention relates to an apparatus and method for reducing backflow in a heat-assisting pump, such as a ventricular assist device.

2. Description of Related Art

In the U.S. alone, there are more than 400,000 patients each year that are diagnosed with debilitating and disabling end-stage congestive heart failure. For many such patients, the use of some type of device to augment the operation of the heart is the only viable alternative to living with the risks of poor circulation, heart failure, and other related problems. Mechanical blood pumps such as ventricular assist devices, or VADs, have been developed to enhance the life span and improve the quality of life for people with weakened heart conditions.

Unfortunately, known VADs have a number of problems. One such problem is the risk of pump failure. Like any mechanical device, the pump of a VAD is susceptible to wear, fatigue, and other operational problems. Furthermore, VADs are typically powered by an exhaustible battery carried by the person with the VAD. Hence, there are many factors that can induce failure of the VAD. Due to the existence of an additional blood flow path through the VAD, pump stoppage presents a hemodynamic risk. A typically VAD, intended to augment aortic flow and arterial pressure when operating normally, may result in considerable retrograde flow if the impeller should stop spinning. Investigators concerned with this hazard have proposed check valves, balloon occluders, and other measures to reduce the risk of harm to the patient during pump stoppage.

The problem with the use of such additional devices is that they add another piece of equipment to the system, thereby adding another potential mode of failure. For example, the check valve may fail or the balloon may not inflate. In the case of the check valve, that valve may stay open for long periods of time, then be required to close in the infrequent event when regurgitant flow occurs. It is difficult to design a check valve that will operate in blood under this type of condition without clotting.

Thus it would be an advantage in the art to provide an apparatus and method capable of providing satisfactory perfusion during pump stoppage in a heart pump, such as a ventricular assist device. Preferably, such a system and method resists backflow without requiring the addition of extra parts that must activate to block the blood flow path through the VAD. Furthermore, such a system and method is preferably easy to integrate into existing VAD designs, with a minimum of extra expense or installation difficulty.

### SUMMARY OF THE INVENTION

The apparatus of the present invention has been developed in response to the present state of the art, and in particular, in response to the problems and needs in the art that have not yet been fully solved by currently available heart pump devices. Thus, it is an overall objective of the present invention to provide a system and method for reducing the risk of backflow through a ventricular assist device during pump failure.

To achieve the foregoing objective, and in accordance with the invention as embodied and broadly described herein in the preferred embodiment, a backflow resistant ventricular assist device (VAD) is provided, together with associated design and implementation methods. According to one configuration, the ventricular assist device comprises a pump, an inflow cannula, and an outflow cannula. The pump is coupled to a ventricle of a heart via the inflow cannula and to an associated blood vessel, such as the aorta or the pulmonary artery, via the outflow cannula. Thus, the pump draws blood from the ventricle and delivers it to the blood vessel to augment the operation of the weakened heart.

The cannulae and the pump may form a flow path from the ventricle to the blood vessel. The reactance of the flow path is generally inversely proportional to the ability of blood under pulsatile pressure to travel retrograde through the ventricular assist device. It is desirable to ensure that backflow through the flow path is small enough that the weakened heart will be able to provide sufficient circulation to maintain survival of the patient in the event of pump failure, at least until he or she can obtain medical attention.

The reactance of the flow path is generally the sum of the reactances of the pump, the inflow cannula, and the outflow cannula. The design of the pump may permit little modification for reactance enhancement. Therefore, the geometry of the cannulae may be utilized to control the reactance of each cannula, and hence the reactance of the flow path as a whole.

Each of the cannulae has a shank portion and a conduit portion. The shank portion has a comparatively smaller outside diameter so that the shank portion can be inserted into the ventricle or blood vessel through a surgically formed opening. The conduit portion is somewhat bendable so that the conduit portion can comfortably extend between the heart or blood vessel and the pump, which may be disposed generally underneath the heart.

Each cannula has a bore designed to permit passage of blood through the cannula. Each cannula may have a number of geometric characteristics, at least one of which is “tuned,” or set at a level selected to provide the desired cannula reactance. The desired cannula reactance is simply that which provides the desired VAD reactance when added to the reactance of the pump and that of the other cannula.

The geometric characteristics may be any or all of the following: the diameter of the bore, the length of the cannula, the cross sectional shape of the bore, and the compliance of the cannula. The compliance of the cannula is generally its ability to expand to store energy, thereby dampening pulsatile flow. The cross sectional shape of the bore, the length of the cannula, and the diameter of the bore also influence the reactance of the cannula. One or more of these geometric characteristics is simply tuned to provide the desired reactance.

It is desirable to provide comparatively high flow path reactance while keeping the resistance comparatively low. The resistance determines the pressure loss during steady state (i.e., nonpulsatile) operation; hence, the higher the resistance, the more power the pump must supply. Therefore, it is desirable to keep the resistance comparatively low. One or more of the geometric characteristics may also be tuned to ensure that the resistance remains low, for example, under a threshold level. The geometric characteristics may be established in such a manner that the cannula reactance is balanced against the cannula resistance.

Reactance is determined by the inertance of the flow path and by the rate of change of the fluid flow rate through the flow path. Calculation of the inertance leads to a ratio of length-to-diameter squared that will provide the necessary minimum inertance. The bore diameter and the cannula length may then be scaled together, according to the ratio, to ensure that the necessary reactance is obtained.

Alternatively, the reactance may be set at the proper level by adjusting other geometric characteristics. For example, the cannula may be made more compliant by adjusting its material, wall thickness, or other properties. Using a non-circular bore shape may also add to the reactance of the cannula. Any other geometric characteristic that provides alteration of the cannula reactance may alternatively or additionally be tuned to provide the desired reactance level.

Tuning of the geometric characteristics may be performed with reference to the total resistance of the VAD. More precisely, the geometric characteristics may be tuned in such a way that the resistance of the VAD does not reach an unacceptable level.

Thus, in the event of pump failure, the incidence of backflow may be reduced considerably via simple, yet deliberate tuning of geometric characteristics of the cannula. Through the use of reactance-based design, backflow reduction may be obtained without adding additional elements, and hence additional failure modes, to the VAD. Hence, the probability that the patient will maintain sufficient circulatory operation to survive the pump failure may be enhanced. These benefits may be obtained while maintaining the operational efficiency of the VAD. These and other features and advantages of the present invention will become more fully apparent from the following description and appended claims, or may be learned by the practice of the invention as set forth hereinafter.

### BRIEF DESCRIPTION OF THE DRAWINGS

In order that the manner in which the above-recited and other features and advantages of the invention are obtained will be readily understood, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 is a front elevation, partially sectioned view of one embodiment of a ventricular assist device according to the invention, coupled to a heart and nearby blood vessel to augment operation of the heart; and

FIG. 2 is a perspective view of the outflow cannula of the ventricular assist device of FIG. 1.

### DETAILED DESCRIPTION OF THE PREFERRED EMBODIMENTS

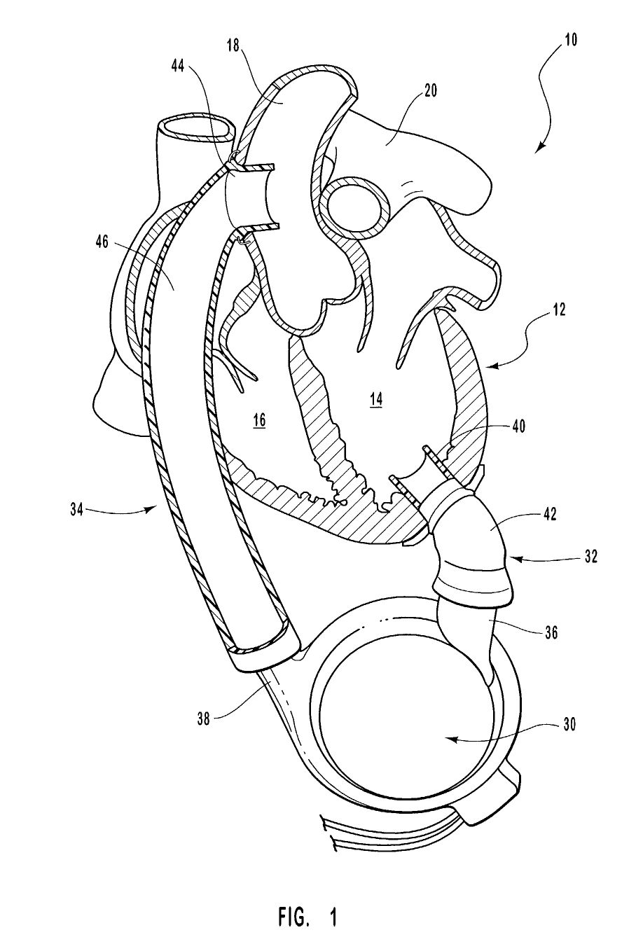

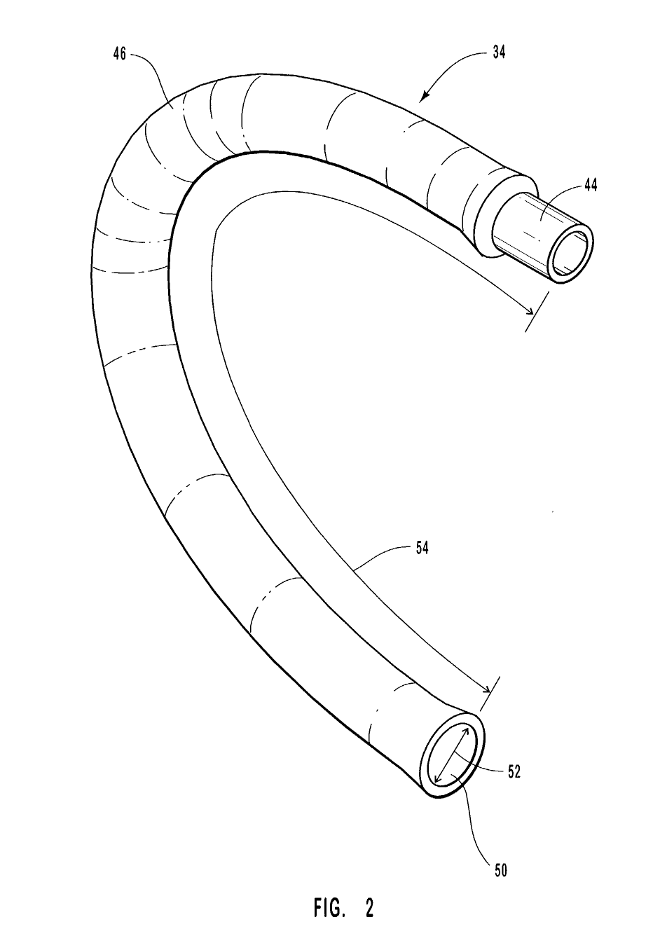

The presently preferred embodiments of the present invention will be best understood by reference to the drawings, wherein like parts are designated by like numerals throughout. It will be readily understood that the components of the present invention, as generally described and illustrated in the figures herein, could be arranged and designed in a wide variety of different configurations. Thus, the following more detailed description of the embodiments of the apparatus, system, and method of the present invention, as represented in FIGS. 1 and 2, is not intended to limit the scope of the invention, as claimed, but is merely representative of presently preferred embodiments of the invention.

The present invention utilizes principles of fluid dynamics to provide a backflow resistant ventricular assist device (VAD) without the use of additional components such as check valves and the like. This is accomplished with a minimum of additional “resistance,” or opposition to continuous flow, so that the size and power requirement of the pump can be kept comparatively low. The “reactance,” or opposition to time-varied flow, on the other hand, is increased to reduce pulsatile flow through the ventricular assist device, such as the backflow that may otherwise be produced when the pump is not functioning.

The higher reactance of the ventricular assist device reduces pulsatile flow in a manner similar to the manner in which shock absorbers dampen vertical motion of an automobile on a bumpy road. The shock absorbers do not generally deflect beyond a steady-state level when the road is not bumpy; however, under bumpy conditions, they absorb the time-varied pressure induced by the bumps to keep the vehicle from bouncing. Similarly, reactance in the ventricular assist device has a minimal impact on steady-state blood flow, yet restricts pulsatile regurgitant flow, or “backflow” through the VAD when the pump is not functioning. The manner in which the present invention utilizes reactance to provide protection against backflow will be described in greater detail with reference to FIGS. 1 and 2, as follows.

Referring to FIG. 1, a front elevation, partially sectioned view depicts one embodiment of a ventricular assist device 10, or VAD 10. The VAD 10 is installed to augment the operation of a heart 12. The heart 12 has a left ventricle 14 and a right ventricle 16. The left ventricle 14 delivers blood to an aorta 18, which conveys the blood throughout the body. The right ventricle 16 delivers blood to a pulmonary artery 20, which conveys the blood to the lungs (not shown).

According to the embodiment of FIG. 1, the VAD 10 has a pump 30, which may be disposed generally underneath the heart 12, as shown. Additionally, the VAD 10 has an inflow cannula 32 and an outflow cannula 34. The VAD 10 is a left ventricular assist device (LVAD) designed to aid the left ventricle 14. Although FIG. 1 depicts an LVAD, the principles of the present invention are equally applicable to right ventricular assist devices (RVADs) and other types of heart pump devices.

As depicted in FIG. 1, the inflow cannula 32 conveys the blood from the left ventricle 14 to the pump 30, and the outflow cannula 34 conveys blood from the pump 30 to the aorta 18. The inflow conduit 32 is coupled to an inflow coupling 36 of the pump 30, while the outflow conduit 34 is coupled to an outflow coupling 38 of the pump 30.

The inflow cannula 32 has a shank portion 40 and a conduit portion 42. The shank portion 40 is inserted partially into the left ventricle 14 via a surgically-formed opening in the wall of the heart 12. The shank portion 40 may be held in place by a sutured cuff, clamp, or the like (not shown). The conduit portion 42 is at least somewhat flexible and conveys blood between the shank portion 40 and the inflow coupling 36 of the pump 30.

Similarly, the outflow cannula 34 has a shank portion 44 and a conduit portion 46. The shank portion 44 is inserted partially into the aorta 18 via a surgically-formed opening in the wall of the aorta 18. The shank portion 44 may also be held in place by some type of fastening device (not shown). The conduit portion 46 is at least somewhat flexible and conveys blood between the outflow coupling 38 of the pump 30 and the shank portion 44.

The pump 30, the inflow cannula 32, and the outflow cannula 34 form a flow path through the VAD 10. The flow path is designed to have a high “reactance,” or opposition to time-varied fluid flows. Preferably, the flow path also has a “resistance,” or opposition to steady state fluid flow, that is below a given threshold. The total pressure drop through the bypass path, between the left ventricle 14 and aorta 18, is the summation of the resistive pressure drop and reactive pressure drop. The former, as the name suggests, is due to the resistance within the flow path, and is totally dissipative. In other words, this drop in pressure is completely lost as wasted energy, or heat. The reactive component of pressure drop is not dissipative; therefore, it is recoverable.

Reactance and resistance are somewhat akin to inductance and resistance in an electric circuit. A resistor draws energy from the circuit in proportion to the electric current, or flow rate of charge through the circuit. Conversely, an inductor stores energy (possibly with some losses) in proportion to the rate of change of the electric current. Hence, the resistor draws energy from any type of current, while an inductor has little effect on a direct current (DC), but may dramatically dampen an alternating current (AC) through the circuit.

Returning to the VAD 10 of FIG. 1, the combination of a comparatively high reactance with limited resistance provides efficient operation as well as backflow protection. Thus, when the pump 30 is operating normally to provide a substantially consistent flow rate of blood through the VAD 10, little pulsing of the blood flow occurs because as the left ventricle 14 is unloaded, the force of its contractions diminishes. The energy losses are comparatively small because the resistance is limited. As a consequence, the pump 30 and its power source may both be comparatively compact. If desired, the VAD 10 may be designed to have a resistance under a target level designed to provide sufficient circulation with the use of known pump and/or cannula designs.

When the pump 30 ceases to operate, the incidence of backflow is reduced by the comparatively high reactance. More precisely, the pulsatility of the blood will increase because the increased diastolic pressure within the left ventricle 14 causes it to beat more forcefully; this is known as the “Frank-Starling Law of the Heart.” This presumes, of course, that the heart maintains a certain ability to contract (known as “contractility”) which is commonly the case, even in patients suffering from heart failure. The increase in the time-varied component of the blood flow causes the structure of the VAD 10 to absorb larger amounts of energy from the blood flow due to the high reactance.

Preferably, the reactance is sufficient to limit backflow enough to permit natural life-sustaining blood circulation in the event of failure of the pump 30. In this application, “natural life-sustaining blood circulation” is circulation sufficient to sustain the life of the patient for the time period immediately after pump failure. Since the VAD 10 is not functioning, the circulation must be provided by the heart 12 alone. Hopefully, within this time period, the patient is able to receive medical attention to repair or replace the pump 30.

As mentioned previously, the reactance and resistance of the flow path of the VAD 10 are summations of the reactances and resistances of the individual components 30, 32, 34 of the VAD 10. Thus, the total reactance and resistance may be adjusted by changing the reactance and resistance of one or more of the components 30, 32, 34. The design of the pump 30 has a comparatively high number of constraints, and may thus not be easily manipulated to adjust the pump reactance or resistance. Hence, one or both of the inflow cannula 32 and the outflow cannula 34 may be uniquely designed to provide the desired reactance and resistance levels. Possible methods of providing the desired cannula reactance and resistance levels will be further described in connection with FIG. 2.

Referring to FIG. 2, a perspective view illustrates the outflow cannula 34 of FIG. 1. The inflow cannula 32 may have a substantially similar configuration. Hence, the following discussion regarding characteristics and tuning of the outflow cannula 34 may readily be applied to the inflow cannula 32. As shown, the outflow cannula 34 has a bore 50 through which the blood travels.

The outflow cannula 34 has a number of geometric characteristics, one or more of which may be “tuned,” or set at a level selected to provide the desired reactance and/or resistance. For example, the geometric characteristics may include a diameter 52 of the bore 50, a length 54 of the outflow cannula 34, a cross sectional shape of the bore 50, and a compliance of the cannula 34. As used herein, “diameter” refers consistently to bore diameter, or inside diameter, as opposed to outside diameter.

The diameter 52 may be uniform along the length of the outflow cannula 34, or may vary. The diameter 52 applies to the conduit portion 46; the bore 50 may have the same diameter in the shank portion 44. Alternatively, the bore 50 of the shank portion 44 may have a smaller diameter, or the bore 50 may be continuously tapered along the length 52. According to certain configurations, the diameter 52 ranges from about 3 millimeters to about 20 millimeters. Furthermore, the diameter 52 may range from about 5 millimeters to about 14 millimeters. Furthermore, the diameter may range from about 7 millimeters to about 10 millimeters. Yet further, the diameter 52 may be about 8 millimeters.

Due to the position of the pump 30, the length 54 of the outflow cannula 34 may be somewhat greater than that of the inflow cannula 32. Hence, the design of the outflow cannula 34 may generally have a proportionately greater impact on the resistance and reactance of the VAD 10. However, both cannulae 32, 34 may be designed concurrently to provide the necessary combined reactance and resistance characteristics. According to certain configurations, the length of the inflow cannula 32 plus the length 54 ranges from about 15 centimeters to about 50 centimeters. Furthermore, combined length may range from about 20 centimeters to about 40 centimeters. Yet further, the combined length may be about 25 centimeters.

In the embodiment of FIG. 2, the cross sectional shape of the bore 50 is circular, as shown. However, different cross sectional shapes, such as polygons, ellipses, splined shapes, and the like may be used to alter the resistance and/or reactance of the cannulae 32, 34. The circular shape may be the simplest to design because there are a wealth of known analytical relationships and equations dealing with flow through a circular bore.

The compliance of the cannulae 32, 34 is generally their ability to expand under pressure. Such expansion provides energy storage to enhance the reactance of the cannulae 32, 34. Compliance may be considered a geometric characteristic because it depends at least in part upon the geometry of the cannulae 32, 34, for example, the thickness of the wall that encircles the bore 50. Compliance is also determined by other considerations such as the material(s) of which the cannulae 32, 34 are formed. According to one example, the compliance of the cannulae 32, 34 ranges from about 0 mL/mmHg to about 5 mL/mmHg. Furthermore, the compliance may range from about 0.10 mL/mmHg to about 2 mL/mmHg. Yet further, the compliance may be about 1 mL/mmHg.

Of course, other geometric characteristics may be adjusted in addition or in the alternative to those mentioned above. Such geometric characteristics may include the surface roughness of the bore 50, the pathway taken by the cannulae 32, 34 (i.e., straight, gradually bent, or elbowed), and any other such characteristics that influence the reactance and/or resistance of the cannulae 32, 34.

As mentioned previously, the reactance of the flow path is preferably sufficient to provide natural life-sustaining blood circulation, while the resistance is preferably sufficiently low to permit the use of a comparatively compact pump 30 and power supply (not shown). This balance between resistance and reactance may be obtained by tuning one of the geometric characteristics until the minimum reactance is met or exceeded without exceeding the maximum resistance. In the alternative, multiple geometric characteristics may be simultaneously tuned to provide the desired resistance and reactance. The diameter 52 and the length 54 may be the easiest to alter because such changes, and their impact on the reactance and resistance, are easy to model mathematically.

Reactance is equal to inertance multiplied by the “pulse rate,” or rate of change of the flow rate of the fluid. As will be shown subsequently, inertance is proportional to the length of the cannulae 32, 34 divided by the square of the diameter of the cannulae 32, 34. This assumes that the bore 50 has a circular cross section. Hence, by constraining the design of the cannulae 32, 34 to maintain a specific length-to-diameter squared ratio, the desired minimum inertance may be obtained. Such a ratio facilitates the design of a range of inflow and outflow cannulae that all provide equal resistance and reactance.

The resistance of the cannulae 32, 34 also depends on their length and diameter, although with a somewhat more complex relationship. A suitable design for the cannulae 32, 34 may be selected based on the desired length 54 and/or the reactance and resistance of the remaining components of the VAD 10, i.e., the pump 30. Based on these factors, a diameter that provides the desired reactance and resistance characteristics may be determined.

According to one method, the VAD 10 is designed by, first, determining the desired overall reactance and resistance for the VAD 10. It may be desirable to set a lower reactance threshold that must be exceeded by the reactance, and an upper resistance threshold that must not be exceeded by the resistance. The resistance and reactance of the pump 30 are then determined through the use of analytical or experimental methods. The resistance and reactance of the pump 30 are then subtracted from the desired resistance and reactance for the VAD 10. The remaining resistance and reactance must then be provided by the cannulae 32, 34.

If desired, the design of only one of the cannulae 32, 34, such as the outflow cannula 34, may be adjusted to provide the desired reactance and resistance. The other cannula 32 or 34 may be of a standard design. In such a case, the reactance and resistance of the standard cannula 32 or 34 may be subtracted from the resistance and reactance obtained above. The result may then be used as the basis for designing the remaining cannula 32 or 34 by altering one or more geometric characteristics, as described above. The remaining cannula 32 or 34 may then be formed with the necessary geometric characteristics, according to any known manufacturing process.

In the alternative, both of the cannulae 32, 34 may be uniquely designed to provide the desired reactance and resistance, in combination with each other. One or more geometric characteristics of each of the cannulae 32, 34 may then be adjusted to obtain the necessary combined reactance and resistance. If desired, length-to-diameter squared ratios may be calculated in the manner described above and utilized to determine the geometric characteristics of the cannulae 32, 34. The cannulae 32, 34 may then be formed with the necessary geometric characteristics, through the use of any known manufacturing process.

If desired, an extra part may be retrofit to an existing VAD design to provide the benefits of the present invention. For example, with reference to the VAD 10, the pump 30 and cannulae 32, 34 may be of a standard configuration. An extra conduit or coupling (not shown) may be added at some point along the flow path to augment the reactance of the VAD 10. Thus, existing VAD systems need not necessarily be redesigned to obtain the benefits of the present invention.

One exemplary design approach for the cannulae 32, 34 will now be provided, with sample values that reflect one possible configuration of the VAD 10. The values presented herein are merely exemplary, and are used to illustrate how one or more cannulae can be designed for backflow resistance utilizing the principles of the invention.

In order to reduce backflow to acceptable levels for a heart operating at a normal rate, but with only about 50% contractile reserve, it may be desirable for the VAD 10 to have an inertance of at least 1.4×10⁷ kg/m⁴. This inertance level is selected to maintain a pump failure arterial pressure of approximately 45 mmHg, which is generally sufficient to sustain life. An inertance value greater than about 1.8×10⁷ kg/m⁴ may be more preferable to provide an even larger margin of safety. Yet more safety may be obtained with inertances as high as, for example, 2.4×10⁷ kg/m⁴, or even 3.0×10⁷ kg/m⁴. For healthier patients, a maximum inertance of about 1.2×10⁷ kg/m^{4,} 1.0×10⁷ kg/m⁴, or even 0.7×10⁷ kg/m⁴ may be sufficient.

The pump 30 itself provides a portion of this inertance, with an amount that varies depending on the type of pump used. For example, the Medquest CF4b VAD pump has an inertance of approximately 1.3×10⁷ kg/m⁴, thereby requiring the cannulae 32, 34 to provide only an additional 5.0×10⁶ kg/m⁴ to obtain the comparatively safe inertance value of 1.8×10⁷ kg/m⁴.

Inertance of a cannula, or L_{c}, is obtained with the equation:

$${L}_{c} = \frac{ρ l}{A}$$

in which

ρ=the density of the fluid, which is about 1,050 kg/m³ for blood,

l=the combined length of the cannulae 32, 34, which is about 10 inches or 0.254 m, and

A=the cross sectional area of the cannulae 32, 34.

For a cannula with a circular cross section, the cross sectional area of the cannula is given by the equation:

$$A = \frac{π {D}^{2}}{4}$$

in which

D=the diameter of the cannula.

Combining the two formulas above and solving for D,

$$D = \sqrt{\frac{4 ρ l}{π {L}_{c}}}$$

After the values above are inserted, with L, set to 5.0×10⁶ kg/m⁴, a value of 8.24 mm is obtained for D. This may be rounded to 8.0 mm to obtain a cannula size that provides the desired total inertance level for a total cannula length of 25.4 centimeters. The ratio of length-to-diameter squared (l/D²) is therefore approximately 4,000. Any set of inflow and outflow cannulae will have the same inertance if the ratio of length-to-diameter squared is the same. For example, if the combined length of the inflow and outflow cannulae were doubled, the maximum diameter of the cannulae would have to be multiplied by the square root of two, or about 1.41. The length-to-diameter squared ratio need not be 4,000, but may range from about 1,000 to about 10,000, from about 2,000 to about 8,000, or from about 3,000 to about 6,000.

If a different pump were used, the inflow and outflow cannulae 32, 34 may be redesigned accordingly to maintain the desired total inertance. Hypothetically, if a pump with no inertance were used, the cannulae 32, 34 would have to provide the entire desired inertance. For example, if the pump 30 provided no inertance, solving the last equation above, using the minimum total inertance value of 1.4×10⁷ kg/m⁴, yields a requirement of about 4.92 mm, or approximately 5 mm, for the diameter 52 of the bore 50 of the cannulae 32, 34.

As mentioned previously, in addition to providing a minimum inertance level for the VAD 10, it is also desirable to ensure that the resistance of the VAD 10 is not too high. For example, it may be desirable to keep the inefficiency, or power loss, of the VAD 10 under about 1 Watt under normal operating conditions. This figure is based the nominal power consumption of a typical VAD system, which is about 10 Watts. Battery life, heat rejection limitations, or other considerations may alternatively dictate the maximum acceptable inefficiency of a VAD; the 1 Watt power loss limitation has been selected to simply provide an efficiency equal to or greater than 90% for the VAD.

Through the use of power loss equations known in the art, it can be shown that this power loss limitation corresponds generally to a maximum resistance of about 4.5 mmHg/lpm for both of the cannulae 32, 34. The maximum resistance need not necessarily be 4.5 mmHg/lpm, but may range, for example, from about 2.5 mmHg to about 10 mmHg/lpm, or from about 3.5 mmHg/lpm to about 7 mmHg/lpm.

Taking both of the cannulae 32, 34, again, as a single tubular vessel, the resistance R_{c} through the cannulae 32, 34 is give by the equation:

$${R}_{c} = \frac{Δ p}{Q}$$

in which

Δp=the pressure drop through the cannulae 32, 34, and

Q=the volumetric flow rate of fluid through the cannulae 32, 34, which is about 5 liters per minute (lpm).

Pressure drop can be determined by the following equation:

Δp=ρghₗ

in which

ρ=the density of the fluid, which is about 1,050 kg/m³ for blood,

g=the gravitational constant, which is about 9.8 m/s², and

hₗ=the head loss through the cannulae 32, 34.

The head loss hₗ is given by the Darcy-Weisbach equation:

$${h}_{l} = f \frac{l}{D} \frac{{v}^{2}}{2 g}$$

in which

f=the friction factor of the cannulae 32, 34,

l=the combined length of the cannulae, which is about 10 inches or 0.254 m,

D=the bore diameter of the cannulae 32, 34, which is about 8 mm, and

v=the velocity of the fluid, and

g=the gravitational constant, which is about 9.8 m/S².

Combining the equations above yields the following:

$$Δ p = f \frac{l}{D} \frac{ρ {v}^{2}}{2}$$

The fluid velocity v is given by the equation

$$v = \frac{Q}{A}$$

in which

Q=the volumetric flow rate of fluid through the cannulae,32, 34, which is about 5 liters per minute (lpm), or 8.33×10⁻⁵ m³/s, and

A=the cross sectional area of the cannulae 32, 34.

As mentioned previously, A is given by the formula:

$$A = \frac{π {D}^{2}}{4}$$

Inserting a value of 8 mm for D yields a value of about 5.03×10−5 m² for A.

Solving the previous equation for v yields a value of about 1.66 m/s.

The friction factor f may be obtained by using Colebrook's formula for the entire nonlaminar range of the Moody Chart:

$$\frac{1}{\sqrt{f}} = - 2.0 log ( \frac{e / D}{3.7} + \frac{2.51}{Re \sqrt{f}} )$$

in which

e=the surface roughness of the bore 50 of the cannulae 32, 34, which is about 0.1 mm, or 100 microns,

D=the bore diameter of the cannulae 32, 34, which is about 8 mm, and

Re is the Reynolds number for the cannulae 32, 34.

Sometimes an approximate formula is used to obtain a closed form solution for f:

$$f = \frac{1.325}{{\{ ln [ ( e / 3.7 D ) + ( 5.74 / {Re}^{0 \; 9} ) ] \}}^{2}}$$

which is valid for cases in which 10⁻⁶<e/D<10⁻² and 5000<Re<10e⁸.

The Reynolds number Re may be obtained by the equation

$$Re = \frac{vD}{η}$$

in which

v=the fluid velocity, which is about 1.66 m/s,

D=the diameter of the cannulae 32, 34, which is about 8 mm, and

η=the viscosity of the fluid (blood), which is about 2.86×10⁻⁶ m²/s.

Solving for Re yields a value of about 4642. Solving, then, for f yields a value of about 0.0515 with the approximate formula, and about 0.0499 with the exact formula. Solving for Δp provides a value of about 2,292 Pascals, which is about 17.2 mmHg. Solving, then, for R yields a value of about 3.44 mmHg/lpm for the resistance through the cannulae 32, 34. This value is within the exemplary maximum value provided above, which is 4.5 mmHg/lpm. Hence, with the Medquest CF4b pump, if the cannulae 32, 34 have a total length of about 25.4 centimeters and an interior diameter of about 8 mm, the desired inertance will be obtained without the presence of excessive resistance.

As mentioned previously, if a pump with a smaller inertance were used, obtaining the desired total inertance would require the use of a smaller cannula diameter. As a result, obtaining the desired inertance without exceeding the maximum desirable resistance would be more difficult. Thus, it is desirable to use a pump with a comparatively high inertance and a comparatively low resistance.

Furthermore, the resistance of the cannulae 32, 34 is sensitive to the surface roughness of the bore 50, denoted by e above. For example, if e were to have a value 1 mm, rather than 0.1 mm, the equations above would yield a friction factor f of about 0.1121 (approximate) or 0.1190 (exact). As a result, the pressure drop Δp through the cannulae 32, 34 would be about 5,430 Pascals, yielding a resistance R of about 8.15 mmHg/lpm. This exceeds the maximum desirable resistance value. Therefore, using a high inertance pump 30 and smooth cannulae 32, 34 makes the desired balance between inertance and reactance easier to obtain.

Through the use of the apparatus and method of the present invention, patients with heart conditions may receive circulatory aid with a diminished risk of serious injury or death in the event of pump failure. By controlling the reactance of a VAD, the incidence of backflow under pump stoppage conditions may be reduced, thereby enhancing the probability that the patient will have adequate circulation for survival until the VAD can be repaired. Such backflow control can be obtained without the use of additional devices that present extra failure modes.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

What is claimed and desired to be secured by United States Letters Patent is:

1. A backflow resistant ventricular assist device comprising:
a pump;
an inflow cannula coupled to the pump to deliver blood to the pump from a ventricle; and
an outflow cannula coupled to the pump to convey blood from the pump to a blood vessel;
wherein the pump, the inflow cannula, and the outflow cannula form a flow path with a reactance sufficient to resist backflow through the flow path during pump stoppage to permit natural life-sustaining blood circulation.

2. The backflow resistant ventricular assist device of claim 1, wherein at least one of the inflow cannula and the outflow cannula has at least one geometric characteristic tuned to provide the reactance.

3. The backflow resistant ventricular assist device of claim 2, wherein the geometric characteristic comprises a diameter of a bore of the cannula.

4. The backflow resistant ventricular assist device of claim 3, wherein the diameter of the bore ranges from about 7 millimeters to about 10 millimeters.

5. The backflow resistant ventricular assist device of claim 3, wherein the diameter is selected to maintain an established length-to-diameter squared ratio.

6. The backflow resistant ventricular assist device of claim 5, wherein the length-to-diameter squared ratio ranges from about 3,000 to about 6,000.

7. The backflow resistant ventricular assist device of claim 2, wherein the geometric characteristic comprises a length of the cannula.

8. The backflow resistant ventricular assist device of claim 2, wherein the geometric characteristic comprises a cross sectional shape of a bore of the cannula.

9. The backflow resistant ventricular assist device of claim 2, wherein the geometric characteristic comprises a compliance of the cannula.

10. The backflow resistant ventricular assist device of claim 2, wherein the flow path has an inertance ranging from about 0.7×10⁷ kg/m⁴ to about 3.0×10⁷ kg/m⁴.

11. The backflow resistant ventricular assist device of claim 2, wherein the geometric characteristic is also tuned to keep a resistance of the flow path under a threshold level.

12. The backflow resistant ventricular assist device of claim 11, wherein the resistance of the flow path ranges from about 2.5 mmHg/lpm to about 10 mmHg/lpm.

13. The backflow resistant ventricular assist device of claim 11, wherein the reactance of the flow path is determined by adding reactances of the inflow cannula, pump, and outflow cannula, and wherein the resistance of the flow path is determined by adding resistances of the inflow cannula, pump, and outflow cannula.

14. A cannula for a backflow resistant ventricular assist device having a pump, the cannula comprising:
a shank portion configured to be inserted at least partially into a body part; and
a conduit portion that conveys blood between the shank portion and a pump;
wherein at least one of the shank portion and the conduit portion has at least one geometric characteristic tuned to provide a reactance of the cannula sufficient to resist backflow through the cannula during pump stoppage to permit natural life-sustaining blood circulation.

15. The cannula of claim 11, wherein the geometric characteristic comprises a diameter of a bore of the conduit portion.

16. The cannula of claim 11, wherein the diameter of the bore ranges from about 7 millimeters to about 10 millimeters.

17. The cannula of claim 11, wherein the diameter is selected to maintain an established length-to-diameter squared ratio.

18. The cannula of claim 17, wherein the length-to-diameter squared ratio ranges from about 3,000 to about 6,000.

19. The cannula of claim 14, wherein the geometric characteristic comprises a length of the cannula.

20. The cannula of claim 14, wherein the geometric characteristic comprises a cross sectional shape of a bore of the conduit portion.

21. The cannula of claim 14, wherein the geometric characteristic comprises a compliance of the cannula.

22. The cannula of claim 14, wherein the reactance of the cannula is determined with reference to reactances of the pump and of a second cannula to provide a desired reactance of the ventricular assist device.

23. The cannula of claim 22, wherein a summation of inertances of the cannula, pump, and second cannula ranges from about 0.7×10⁷ kg/m⁴ to about 3.0×10⁷ kg/m⁴.

24. The cannula of claim 14, wherein the geometric characteristic is also tuned to keep a resistance of the cannula under a threshold level.

25. The cannula of claim 24, wherein the resistance of the cannula is determined with reference to resistances of the pump and of a second cannula to ensure that the ventricular assist device has a resistance less than a maximum resistance.

26. The cannula of claim 25, wherein a summation of the resistances of the cannula, pump, and second cannula ranges from about 2.5 mmHg/lpm to about 10 mmHg/lpm.

27. A method for reducing an incidence of backflow through a ventricular assist device comprising a pump configured to be coupled to a ventricle and a blood vessel through the use of at least one cannula, the method comprising:
calculating a desired cannula reactance selected to provide a reactance of the ventricular assist device that is sufficient to resist backflow through the ventricular assist device during pump stoppage to permit natural life-sustaining blood circulation;
tuning at least one geometric characteristic of the cannula to provide the desired cannula reactance; and
forming a cannula having the geometric characteristic.

28. The method of claim 27, wherein tuning the geometric characteristic comprises determining a diameter of a bore of the cannula.

29. The method of claim 28, wherein determining the diameter of the bore comprises selecting a diameter ranging from about 7 millimeters to about 10 millimeters.

30. The method of claim 28, wherein sizing the diameter comprises maintaining an established length-to-diameter squared ratio.

31. The method of claim 30, wherein sizing the diameter comprises maintaining a length-to-diameter squared ratio ranging from about 3,000 to about 6,000.

32. The method of claim 27, wherein tuning the geometric characteristic comprises determining a length of the cannula.

33. The method of claim 27, wherein tuning the geometric characteristic comprises selecting a cross sectional shape of a bore of the cannula.

34. The method of claim 27, wherein tuning the geometric characteristic comprises determining a compliance of the cannula.

35. The method of claim 27, wherein tuning the geometric characteristic comprises providing a total inertance of the ventricular assist device ranging from about 0.7×10⁷ kg/m⁴ to about 3.0×1⁰⁷ kg/m⁴.

36. The method of claim 27, wherein tuning the geometric characteristic comprises keeping a resistance of the ventricular assist device under a threshold level.

37. The method of claim 36, wherein tuning the geometric characteristic comprises providing a resistance of the ventricular assist device ranging from about 2.5 mmHg/lpm to about 10 mmHg/lpm.

38. The method of claim 27, wherein calculating the desired cannula reactance comprises:
determining the desired reactance of the ventricular assist device;
determining a reactance of the pump; and
subtracting the reactance of the pump from the desired reactance of the ventricular assist device.

39. The method of claim 38, wherein the ventricular assist device is further configured to be coupled to the ventricle and the blood vessel through the use of a second cannula, the method further comprising:
forming the second cannula, the second cannula having the geometric characteristic.
